(19) United States
(12) Patent Application Publication
(10) Pub. No.: US 2003/0109582 A1
(43) Pub. Date: Jun. 12, 2003

(54) Methods and compositions for stimulating secretions from paneth cells

(76) Inventor: Michael A. Zasloff, Merion, PA (US)

Correspondence Address: HENRY E. MILLSON, JR., 675 GOLDEN HAWK DRIVE, PRESCOTT, AZ, 86301, US

(21) Appl. No.: 10/313,261

(22) Filed: Dec. 6, 2002

Related U.S. Application Data
(60) Provisional application No. 60/337,824, filed on Dec. 10, 2001.

(51) Int. Cl. A61K 31/198

(52) U.S. Cl. 514/561

(57) ABSTRACT

Methods and compositions for stimulating Paneth cells to release natural antimicrobial agents including peptides, to reduce or eliminate pathogenic organisms in the GI tract of mammalian bodies, including humans, utilizing an active isoleucine compound as a secretagogue.

### CROSS REFERENCE TO RELATED APPLICATIONS

This application claims the benefit of copending provisional application serial No. 60/337,824, filed Dec. 10, 2001.

### FIELD OF THE INVENTION

This invention relates to methods and compositions for stimulating the secretion of anti-pathogenic organism compounds from Paneth cells of the gastrointestinal tract in animal bodies.

### BACKGROUND OF THE INVENTION

From time to time pathogenic organisms, i.e. those that can cause disease, enter the upper gastrointestinal (GI) tract of the animal body, usually through food and water, passing through the stomach and upper GI tract. Classic examples are the microorganisms that cause dysentery, such as species of the bacteria Shigella and Salmonella, viruses such as those of the Rotavirus family and parasites such as Crytosporidium. In addition, organisms such as poliovirus enter the GI tract but cause diseases at sites elsewhere in the body.

Current treatments that deal with reducing the threat of infection by infectious agents entering the body through the GI tract include sterilization of food and water, prophylaxis with oral antibiotics, and vaccination.

### SUMMARY OF THE INVENTION

This invention relates to methods and compositions for stimulating Paneth cells to release secretions containing natural antimicrobial agents, including peptides, to reduce or eliminate pathogenic organisms in the GI tract of animal bodies, especially in human and other mammalian bodies. It has been discovered that the essential amino acid isoleucine and its active isomers and derivatives act as specific Paneth cell secretagogues, which significantly enhance the body's immune response, and are safe, chemically stable compounds.

### DETAILED DESCRIPTION OF THE INVENTION

Other than in the operating examples, or where otherwise indicated all numbers expressing quantities of ingredients used herein are to be understood as modified in all instances by the term “about”.

The present invention exploits the fact that the animal body stores large quantities of potent antimicrobial substances, in certain anatomic locations, strategically located to destroy a wide spectrum of infective agents attempting to invade the body via the GI tract.

The gastrointestinal tract of mammals is covered by a continuous sheet of epithelial cells that is folded into vilus projections and crypts. Within the base of the crypts, where the stem cells of the GI tract can be found, there are specialized, granular cells called Paneth cells. Both enterocytes and Paneth cells produce antimicrobial peptides. The enterocytes synthesize and secrete antimicrobial peptides both constitutively and upon induction, and either secrete them onto their surface as in the respiratory tract, or as in the rectum, retain them in a cell associated fashion in the superficial non-viable sheets of epithelium. The Paneth cells at the base of the intestinal crypts, in contrast, secrete alpha-defensins into the cryptal well, following a microbial stimulus, resulting in concentrations estimated at mg/ml levels, which eventually flush into the gut lumen.

Both systems contribute to bowel health. In children and adults suffering from diarrhea caused by Shigella, synthesis of the colonic enterocyte beta-defensin HBD-1 and the cathelicidin LL37 is markedly depressed; expression recovers in time during resolution of the illness. Similarly, mice lacking the proteolytic enzyme required for processing cryptins, the murine Paneth cell alpha-defensins, and consequently, lacking functional cryptins, exhibit increased susceptibility to orally administered Salmonella.

The use of the compositions and methods of the present invention result in the stimulation of the Paneth cells of the gastrointestinal tract of man and other mammals to secrete large quantities of naturally occurring broad-spectrum antimicrobial agents, including antimicrobial peptides such as defensins, cryptins, LL37, HBD1, and HBD2, and other antimicrobial agents such as lysozyme, transferrin, lactoferrin, phospholipases, and SLIPI. The substances stored by the Paneth cells exhibit activity against a wide range of infectious agents including bacteria, protozoa, viruses, and fungi.

Heretofore, only a limited number of Paneth cell secretagogues were known, comprising microbes such as fungi and bacteria, and cholinergic agents, such as methacholine. None of the above secretagogues have the properties or the specificity to be used widely and safely. For example, the drug methacholine has complex pharmacological effects associated with cholinergic agents, which include slowing of the heartbeat, dry mouth, anxiety, visual disturbance, and gait disturbances. The use of fungi or bacteria, either live or killed as therapeutic agents is associated with problems of storage, infectivity, incompatibility with foods, or certain toxicology.

It is of course of great medical utility to be able to safely stimulate the Paneth cells of the GI tract on a repeated basis to cause the Paneth cells to discharge their contents into the bowel lumen. Such therapy can rid the bowel of infectious agents which are susceptible to the killing action of the antimicrobial agents stored in the Paneth cells.

The methods and compositions of the present invention utilize isoleucine compounds as specific Paneth cell secretagogues to prevent or treat infectious diseases and other conditions such as Crohn's disease that require the control or elimination of organisms in the GI tract.

L(+)isoleucine is an essential amino acid, having the general formula CH₃CH₂CH(CH₃)CH(NH₂)COOH. An essential amino acid is an amino acid which is critical for normal health and nutrition and cannot be synthesized within the body but must be obtained from an external source, usually via food intake.

While small quantities of L(+)isoleucine are present in some foods and feedstuffs, such quantities present in a typical mammalian diet are insufficient to act as an effective Paneth cell secretagogue.

When used herein, the following terms are to be understood to have the following meanings:

“Paneth cells”: Large sector-shaped cells found at the base of the invaginations of the mammalian gastrointestinal tract (crypts). These cells have been shown to play a role in the control of microbial growth in the GI tract of man and other mammals.

“Secretagogue”: A substance that causes a cell to discharge its stored contents in a physiological fashion, including repeated discharge if this is the nature of the response.

“Isoleucine compound”: Isoleucine, active stereoisomers thereof, and active derivatives thereof, including isoleucine derivatives that are converted by the mammalian body to an active form of isoleucine. Isoleucine stereoisomers include L(+)isoleucine, which is the naturally occurring form, DL-isoleucine, which is a racemic mixture and is the synthetic form, D(−)-allo-isoleucine, and L(+)-allo-isoleucine, although the latter two compounds are much less active. Active derivatives of isoleucine include, but are not limited to, alpha-keto-methyl valerate, isoleucine hydroxamate, and isoleucine butyrate.

The compositions of the invention are pharmacologically acceptable compositions comprising:

A) a Paneth cell secretagogue-effective quantity of at least one pharmacologically active isoleucine compound;

B) at least one pharmacologically acceptable carrier material; and optionally,

C) at least one additional pharmacologically active substance.

In component A), the Paneth cell secretagogue-effective quantity is generally in the range of from 100 mg to 10 grams or more, e.g. up to 50 grams, preferably from 250 mg to 5 grams, of isoleucine compound. The secretagogue-effective quantity will of course vary according to the degree of activity of the isoleucine compound or compounds used as component A) in the above compositions.

Component B) can be one or more of a carrier material such as water, ethanol, oils, including those of petroleum, animal, vegetable or synthetic origin, such as peanut oil, soybean oil, mineral oil, sesame oil, and the like, and excipients such as methylcellulose, carageenan, and the like, and/or other pharmacologically compatible substances. Component B) can also be a food product, e.g. yogurt, pudding, spreadable food product, baked product, drink, soup, gum, candy, dairy product, infant formula, or baby food. Where the composition is used to treat other mammals such as agricultural animals, companion pets, zoo animals, and the like, component B) can be an animal food or feed, such as those containing one or more of chicory, fiber, live probiotic bacteria, silage, grain, corn, soybeans, and wheat.

Optional component C) can be another substance or substances that are effective in promoting gastrointestinal health or which act as additional antimicrobial agents, such as anti-diarrheal agents, e.g. kaolin/pectin compositions, activated charcoal compositions, compositions containing Lactobacillus acidophilus and L bulgaricus, bismuth subsalicylate compositions, activated attapulgite compositions, loperamide compositions, enzyme compositions, and difenoxin/atropine compositions; antibiotics; vitamins; peppermint oil or extract; menthol; quassia; bistort; ginger; angelica; bayberry; chamomile; fish oil; a fatty acid; an omega-3 fatty acid; fiber; flaxseed; slippery elm; niacin; a plant extract; garlic or garlic extract; calcium; stannol esters; lutein; zeaxanthin; cryptoxanthin; isoflavone; an anti-inflammatory compound such as aspirin, ibuprofen, and acetaminophen, and the like.

The invention also includes a composition as described above comprising an infant formula or food containing milk products and/or soy products and an active isoleucine compound.

When the above compositions are in the form of an oral pharmaceutical dosage form, such dosage forms include dry powders, pastes, solutions, gels, tablets, lozenges, capsules, and boluses. The dosage forms can be immediate release or delayed release compositions. The above dosage forms can be readily formulated using excipients and procedures well known for preparing such dosage forms.

One method of the invention is carried out by orally administering one or more isoleucine compounds in a Paneth cell secretagogue-effective quantity to a mammal, wherein the isoleucine compound(s) can be administered as a powder, as a solution in water, or in one of the compositions of the invention.

Another method of the invention is carried out by introducing a composition containing at least one isoleucine compound into the lower bowel, e.g. by means of a suppository or enema containing a Paneth cell secretagogue-effective quantity of the isoleucine compound. Suppository compositions will of course include suppository excipients such as coconut and/or palm kernel oil triglycerides, butylated hydroxyanisole, and the like.

The secretagogue-effective dosages of isoleucine compounds as discussed above for the compositions of the invention can range from 100 mg to 50 grams, preferably from 100 mg to 10 grams, and more preferably from 250 mg to 5 grams.

In one preferred embodiment, one or more active isoleucine compounds can be administered alone, or as a major constituent of a palatable composition, by ingestion at a concentration that results in the “bathing” of the bowel wall with effective concentrations of the isoleucine secretagogue. Preferably the intake should result in concentrations of isoleucine reaching the gut limning cells in excess of 1 mg/ml. This can be accomplished by ingestion of a concentrated solution of isoleucine or a preparation consisting of the powdered substance. The powdered form can be incorporated into a timed release or delayed release pharmaceutical preparation, common to the art, to deliver the isoleucine to more distant portions of the GI tract.

For mammals other than humans, the above oral doses can be readily adjusted according to the size of the animal and the number of Paneth cells present in the animal.

The use of isoleucine compounds in accordance with the invention results in secretions from the Paneth cells that provide potent control, destruction, and prevention of pathogenic microorganisms in the GI tract. As discussed above, such secretions are naturally stored in Paneth cells for the purpose of host defense.

It should be pointed out that the approach provided by the invention is pharmacologically distinct from the induction of the transcription of a gene. Inducers of transcription are substances that are designed to “turn a gene on” and thereby stimulate production of a gene product. This process is generally slow and requires hours to days to become effective. The eventual discharge of the induced gene product falls under the control of other substances and circumstances, and might not be released in either the location, appropriate time, or in the magnitude required. Inducers and secretagogues operate by different mechanisms and exhibit different kinetics of action. In the case of a secretagogue the effects occur within minutes and are generally “post-transcriptional”, involving cellular processes that result in the release of cellular substances stored or readily available for secretion.

The methods of the present invention can be regarded as “indirect” means by which broad spectrum antimicrobial agents can be introduced into the gut lumen, using the body's own defense in place of an exogenous foreign agent.

Examples of disease states and conditions that can be treated by the compositions and methods of the invention include, but are not limited to, traveler's diarrhea, endemic diarrhea, dysentery, viral gastroenteritis, parasitic enteritis, Crohn's disease, ulcerative colitis, irritable bowel syndrome, precancerous states of the gastrointestinal tract, cancer of the gastrointestinal tract, diverticulitis, post-antibiotic diarrhea, Clostridium difficile colitis, lactose intolerance, flatulence, gastritis, esophagitis, heartburn, gastric ulcer, ulcers associated with Helicobacter pylori, duodenal ulcer, short bowel syndrome, dumping syndrome, gluten enteropathy, food intolerance, and used following surgery, immune ablation, or chemotherapy.

As discussed above, the methods and compositions of the present invention have application in the treatment of both human and other mammals, including veterinary and animal husbandry applications for companion animals, farm animals, and ranch animals.

The invention will be illustrated but not limited by the following examples.

### EXAMPLES

### Example 1

An assay was developed utilizing Human Paneth cells, obtained from biopsy material. Substances were screened in vitro, by exposing the isolated human tissue material briefly, and assessing the release of antibiotic activity.

A human Paneth cell secretion assay was developed using isolated intact human crypts from ileum obtained after bowel surgery, following published techniques. About 700 isolated crypts obtained by disruption with EDTA, as estimated using a hemocytometer, were transferred into microfuge tubes. Crypts were then incubated in minimal essential media containing non-essential amino acids, with or without various substances (such as isoleucine) in a total volume of 0.5 ml for 30 min or 1 hour at 37° C. After incubation the tissue suspension was spum, and the supernatants collected 10 microliter aliquots were then mixed with either 500 or 1000 colony forming units (cfu) of Salmonella typhimurium phoP- and incubated for 1 hour at 37° C. Surviving bacteria were quantitated by plating on nutrient agar. Bactericidal assays were conducted in triplicate. Data were expressed as the percent bacteria killed under experimental conditions relative to those recovered from controls.

Results

Bactericidal activity in secretions against S. typhimurium was detected when crypts were exposed to L(+)isoleucine at 250 microgram/ml for 1 hour (TABLE 1).

TABLE 1

| Conditions | Surviving Bacteria | % Bacterial Cell Killing |
|---|---|---|
| Unexposed bacteria | 386 ± 106 | — |
| Crypts + isoleucine | 140 ± 23 | 63.7 |
| Crypts − isoleucine | 328 ± 82 | 15.0 |

The concentration dependence of isoleucine-induced secretion was determined (TABLE 2). The assay was conducted as in TABLE 1, but in the presence of varying concentrations of isoleucine for 30 minutes.

TABLE 2

| Isoleucine Concentration (μg/ml) | % Bacterial Cell Killing |
|---|---|
| 0 | 2.65 |
| 5 | 4.1 |
| 50 | 22.5 |
| 500 | 51.7 |

As shown in the above example, isoleucine acts to cause human Paneth cells to release substances that kill a typical enteric organism, Salmonella typhimurium. The effect is dose dependent.

### Example 2

L-isoleucine was administered to nine volunteer Crohn's disease patients with mild abdominal symptoms. Four grams/day of powdered L-isoleucine divided into two doses was orally administered for 4 weeks. The CDAI (Crohn's disease activity index—Best W, Beckel J, Singleton J, Kern F J, Development of a Crohn's Disease Activity Index: National Cooperative Crohn's Disease Study. Gastroenterology 1976; 70:439-44) was calculated at the beginning and following the fourth week of the L-isoleucine administration period. All medication regiments were fixed throughout the study period.

The frequency of diarrhea gradually decreased, and stool consistency improved; the mean CDAI at fourth week was significantly improved (from a mean CDAI of 120 to a mean CDAI of 90).

### Example 3

The population density of viable Lactobacilli in the stools of a group of 9 hospitalized individuals with Crohn's disease was measured before and after a course of L-isoleucine administration. Subjects in this group had been supported nutritionally exclusively by total parenteral nutrition (TPN) for more than one month. One week after a low-residual formula diet was introduced, L-isoleucine was added to the diet (dosed as in Example 2) during the fmal week of hospitalization. Yogurt and other fermented products were restricted as were antibiotics. The mean concentration of fecal Lactobacilli was significantly increased after 1 week of L-isoleucine administration.

The results shown in Examples 2 and 3 show that the stimulation of the Paneth cells in the GI tract using an active isoleucine compound represents a method of therapeutic intervention in the treatment of Crohn's disease and related Paneth cell secretion-sensitive microorganism—caused disorders of the GI tract.

What is claimed is:

1. A method for eliciting antimicrobial secretions from Paneth cells of the gastrointestinal tract in a mammalian body comprising contacting the Paneth cells with a secretagogue-effective quantity of at least one active isoleucine compound.

2. The method of claim 1 wherein the at least one active isoleucine compound is selected form the group consisting of L(+)isoleucine, DL-isoleucine, D(−)-allo-isoleucine, L(+)-allo-isoleucine, and active derivatives of the foregoing.

3. The method of claim 1 wherein the at least one active isoleucine compound is administered orally.

4. The method of claim 1 wherein the at least one active isoleucine compound is administered to the lower bowel.

5. The method of claim 2 wherein the at least one active isoleucine compound is L(+)isoleucine.

6. The method of claim 2 wherein the at least one active isoleucine compound is DL-isoleucine.

7. The method of claim 1 wherein the mammalian body is a human body.

8. The method of claim 7 wherein said effective quantity is in the range of from about 100 mg to about 50 grams.

9. The method of claim 7 wherein said effective quantity is at least 250 mg.

10. A method for treating or preventing infection by pathogenic microorganisms in the gastrointestinal tract of a mammalian body by administering to Paneth cells contained therein a secretagogue-effective quantity of at least one active isoleucine compound.

11. The method of claim 10 wherein the pathogenic organisms are bacteria, viruses, fungi, and/or protozoa.

12. The method of claim 10 wherein the method is used to treat traveler's diarrhea, endemic diarrhea, dysentery, viral gastroenteritis, parasitic enteritis, Crohn's disease, ulcerative colitis, irritable bowel syndrome, precancerous states of the gastrointestinal tract, cancer of the gastrointestinal tract, diverticulitis, post-antibiotic diarrhea, Clostridium difficile colitis, lactose intolerance, flatulence, gastritis, esophagitis, heartburn, gastric ulcer, ulcers associated with Helicobacter pylori, duodenal ulcer, short bowel syndrome, dumping syndrome, gluten enteropathy, or food intolerance.

13. The method of claim 10 wherein the method is used following surgery, immune ablation, or during or following chemotherapy.

14. The method of claim 10 wherein the mammalian body is a human body.

15. The method of claim 10 wherein the mammalian body is a companion animal or a farm or ranch animal.

16. An oral dosage form for administration to a mammalian body comprising:
A) a Paneth cell secretagogue-effective quantity of at least one pharmacologically active isoleucine compound;
B) at least one pharmacologically acceptable carrier material; and optionally,
C) at least one additional pharmacologically active substance.

17. The oral dosage form of claim 16 wherein in component A) the at least one active isoleucine compound is selected from the group consisting of L(+)isoleucine, DL-isoleucine, D(−)-allo-isoleucine, L(+)-allo-isoleucine, and active derivatives foregoing.

18. The oral dosage form of claim 16 wherein in component A) the Paneth cell secretagogue-effective quantity is in the range of from about 100 mg to about 50 grams.

19. The oral dosage form of claim 18 wherein said effective quantity if at least 250 mg.

20. The oral dosage form of claim 16 wherein component A) is L(+)isoleucine.

21. The oral dosage form of claim 16 wherein component A) is DL-isoleucine.

22. The oral dosage form of claim 16 wherein the composition is in the form of a dry powder, a paste, a solution, a gel, a tablet, a lozenge, or a capsule.

23. The oral dosage form of claim 16 wherein component B) is a comestible.

24. The oral dosage form of claim 23 wherein component B) is selected from the group consisting of yogurt, a pudding, a baked product, a drink, a spreadable food product, a soup, gum, candy, a dairy product, an infant formula, or a baby food.

25. The oral dosage form of claim 23 wherein the dosage form is for a mammal other than a human.

26. The oral dosage form of claim 25 wherein the comestible is a companion animal food.

27. The oral dosage form of claim 25 wherein the comestible is an agricultural animal food or feed or a zoo animal food or feed.

28. The oral dosage of claim 16 wherein component C) is present.

29. The oral dosage form of claim 28 wherein component C) is a substance that is effective in promoting gastrointestinal health or which acts as an additional antimicrobial agent.

30. The oral dosage form of claim 28 wherein component C) is an anti-diarrheal agent.

31. A dosage form for rectal administration to a mammalian body comprising:
A) a Paneth cell secretagogue-effective quantity of at least one pharmacologically active isoleucine compound;
B) excipients for the dosage form; and optionally,
C) at least one additional pharmacologically active substance.
